(19) United States
(12) Patent Application Publication
(10) Pub. No.: US 2018/0179191 A1
(43) Pub. Date: Jun. 28, 2018

(54) SYNTHESIS OF A COMPOUND THAT MODULATES THE ACTIVITY OF BROMODOMAIN-CONTAINING PROTEINS

(71) Applicant: Gilead Sciences, Inc., Foster City, CA (US)

(72) Inventors: Jinyu Shen, Edmonton (CA); Mustapha St. Jules, Alberta (CA); David Sperandio, Palo Alto, CA (US); Sankar Mohan, Alberta (CA)

(21) Appl. No.: 15/809,296

(22) Filed: Nov. 10, 2017

Related U.S. Application Data
(60) Provisional application No. 62/425,176, filed on Nov. 22, 2016.

(51) Int. Cl. C07D 403/14; C07D 235/08; C07D 213/30; C07D 261/08; C07B 37/04; C07B 37/10

(52) U.S. Cl. CPC C07D 403/14; C07D 235/08; C07B 37/10; C07D 261/08; C07B 37/04; C07D 213/30

(57) ABSTRACT

The present disclosure provides processes for the preparation of a compound of formula I:

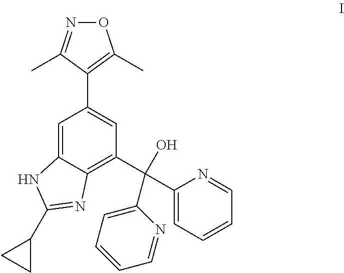 [SMILES: Cc1noc(C)c1-c1cc(C(O)(c2ccccn2)c2ccccn2)c2nc(C3CC3)[nH]c2c1]

or a salt or co-crystal thereof, that modulates the activity of bromodomain-containing proteins. The disclosure also provides compounds and processes for the preparation of the compounds that are synthetic intermediates to the compound of formula I.

### CROSS-REFERENCE TO RELATED APPLICATION

This application claims the benefit of U.S. Application No. 62/425,176, filed on Nov. 22, 2016, which is hereby incorporated by reference in its entirety.

### FIELD

The present disclosure relates generally to the field of organic synthetic methodology for the preparation of compounds that modulate the activity of bromodomain-containing proteins and their synthetic intermediates.

### BACKGROUND

Therapeutic agents that function as modulators or inhibitors of the bromodomain and extraterminal (BET) family of proteins (e.g., including BRD2, BRD3, BRD4, and BRDT) have the potential to remedy or improve the lives of patients in need of treatment for diseases or conditions such as neurodegenerative, cardiovascular, inflammatory, autoimmune, renal, viral and metabolic disorders. In particular, BET modulators or inhibitors have the potential to treat cancer (including carcinoma, lymphoma, multiple myeloma, leukemia, neoplasms or tumors), rheumatoid arthritis, osteoarthritis, atherosclerosis, psoriasis, systemic lupus erythematosus, multiple sclerosis, inflammatory bowel disease, asthma, chronic obstructive airways disease, pneumonitis, dermatitis, alopecia, nephritis, vasculitis, Alzheimer's disease, hepatitis, primary biliary cirrhosis, sclerosing cholangitis, and diabetes (including type I diabetes) among others. The compound named, (2-cyclopropyl-6-(3,5-dimethylisoxazol-4-yl)-1H-benzo[d]imidazol-4-yl)di(pyridin-2-yl)methanol, is effective for treating subjects suffering from or at risk of a disease or condition responsive to the modulation or inhibition of bromodomain-containing proteins. Suitable compounds, including benzimidazole derivatives, for the treatment of such diseases and conditions are disclosed in U.S. Publication No. 2014-0336190, the disclosure of which is incorporated herein by reference in its entirety.

There remains a need in developing new versatile and facile processes for the efficient preparation of benzimidazole derivatives and other similar molecules that modulate or inhibit bromodomain-containing proteins.

### SUMMARY

In one embodiment, this disclosure provides a process for making a compound of formula I, named (2-cyclopropyl-6-(3,5-dimethylisoxazol-4-yl)-1H-benzo[d]imidazol-4-yl)di(pyridin-2-yl)methanol:

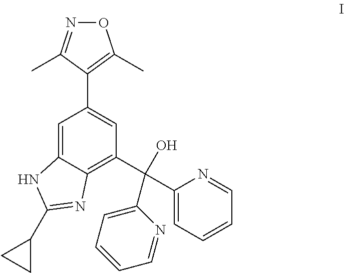 [SMILES: Cc1noc(C)c1-c1cc(C(O)(c2ccccn2)c2ccccn2)c2nc(C3CC3)[nH]c2c1]

or a salt or co-crystal thereof.

In another embodiment, this disclosure provides a process for making a compound of formula II (compound I.H₃PO₄), named (2-cyclopropyl-6-(3,5-dimethylisoxazol-4-yl)-1H-benzo[d]imidazol-4-yl)di(pyridin-2-yl)methanol phosphate complex.

In another embodiment, this disclosure provides a process for preparation of a compound of formula VII:

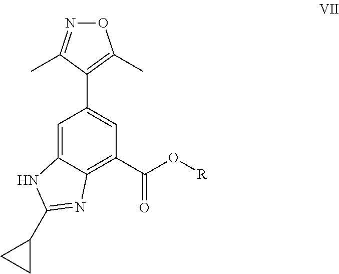

or a salt or co-crystal thereof, comprising contacting a compound of formula V or a salt or co-crystal thereof with a compound of formula VI or a salt or co-crystal thereof:

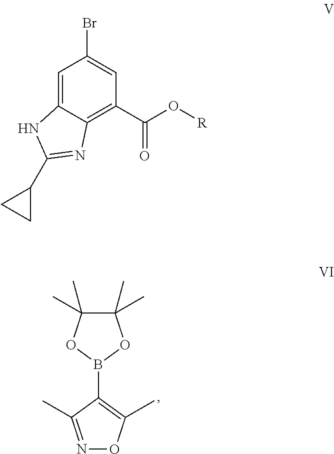

to provide the compound of formula VII or a salt or co-crystal thereof, wherein R is C₁₋₆ alkyl.

In another embodiment, this disclosure provides a process for preparation of a compound of formula I:

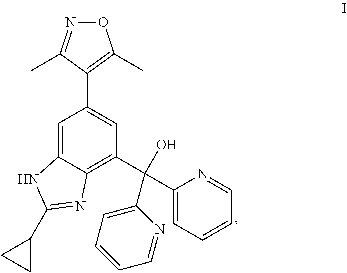 [SMILES: Cc1noc(C)c1-c1cc(C(O)(c2ccccn2)c2ccccn2)c2nc(C3CC3)[nH]c2c1]

comprising:
(a) contacting a compound of formula III or a salt or co-crystal thereof, with a compound of formula IV, or a salt or co-crystal thereof:

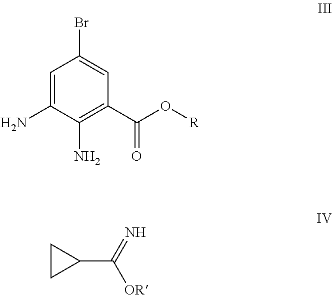

to provide a compound of formula V:

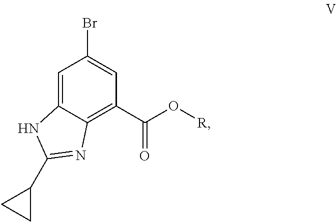

or a salt or co-crystal thereof, wherein R and R′ are independently C₁₋₆ alkyl;
(b) contacting the compound of formula V or a salt or co-crystal thereof with a compound of formula VI:

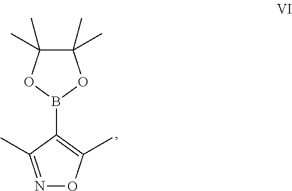

or a salt or co-crystal thereof, to provide a compound of formula VII:

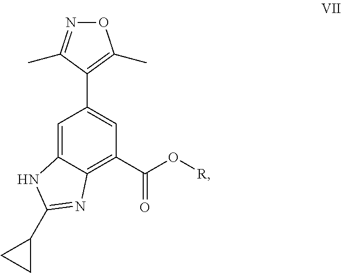

or a salt or co-crystal thereof; and
(c) contacting the compound of formula VII or a salt or co-crystal thereof with a compound of formula VIII:

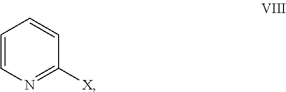

or a salt or co-crystal thereof, wherein X is halo, to provide the compound of formula I.

In another embodiment, this disclosure provides a process for preparation of a compound of formula II (compound I.H₃PO₄), named as (2-cyclopropyl-6-(3,5-dimethylisoxazol-4-yl)-1H-benzo[d]imidazol-4-yl)di(pyridin-2-yl)methanol phosphate complex, comprising
(a) contacting a compound of formula III or a salt or co-crystal thereof, with a compound of formula IV or a salt thereof:

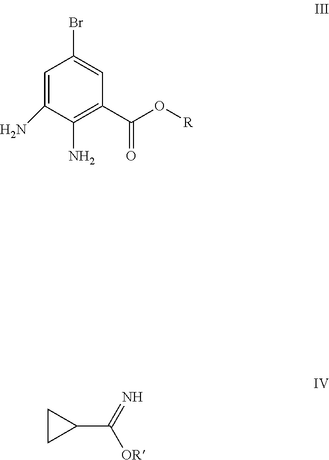

to provide a compound of formula V:

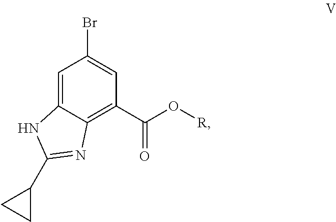

or a salt or co-crystal thereof, wherein R and R′ are indendently C₁₋₆ alkyl;
(b) contacting the compound of formula V or a salt or co-crystal thereof with a compound of formula VI:

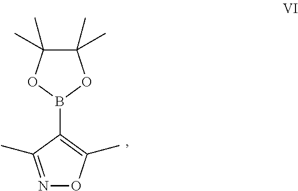

or a salt or co-crystal thereof, to provide a compound of formula VII:

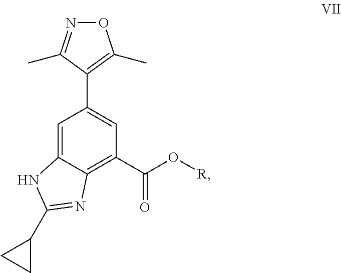

or a salt or co-crystal thereof;
(c) contacting the compound of formula VII or a salt or co-crystal thereof with a compound of formula VIII:

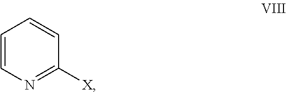

or a salt or co-crystal thereof, wherein X is halo, to provide the compound of formula I:

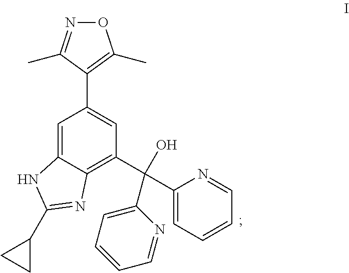 [SMILES: Cc1noc(C)c1-c1cc(C(O)(c2ccccn2)c2ccccn2)c2nc(C3CC3)[nH]c2c1]

and
(d) contacting the compound of formula I with phosphoric acid to provide the compound of formula II.

In another embodiment, this disclosure provides a process for preparation of a compound of formula II (compound I.H₃PO₄), named as (2-cyclopropyl-6-(3,5-dimethylisoxazol-4-yl)-1H-benzo[d]imidazol-4-yl)di(pyridin-2-yl)methanol phosphate complex, comprising
(a) contacting a compound of formula IX or a salt or co-crystal thereof, with cyclopropylcarbaldehyde:

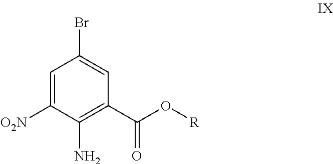

to provide a compound of formula V:

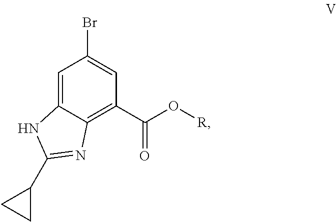

or a salt or co-crystal thereof, wherein R is C₁₋₆ alkyl.

Additional embodiments are described throughout.

### DETAILED DESCRIPTION

The compound, (2-cyclopropyl-6-(3,5-dimethylisoxazol-4-yl)-1H-benzo[d]imidazol-4-yl)di(pyridin-2-yl)methanol, designated herein as compound I, has the following formula:

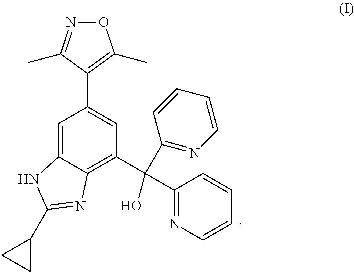 [SMILES: Cc1noc(C)c1-c1cc(C(O)(c2ccccn2)c2ccccn2)c2nc(C3CC3)[nH]c2c1]

Compound I is a selective and potent inhibitor of BET proteins. The synthesis and method of use thereof is described in U.S. Patent Application Publication No. 2014/0336190 A1, which is incorporated herein by reference in its entirety.

Compound II is (2-cyclopropyl-6-(3,5-dimethylisoxazol-4-yl)-1H-benzo[d]imidazol-4-yl)di(pyridin-2-yl)methanol phosphate complex.

In one embodiment, this disclosure provides a process of making compounds of formula I and II.

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cu-v” indicates that the following group has from u to v carbon atoms. For example, “C₁₋₆ alkyl” indicates that the alkyl group has from 1 to 6 carbon atoms. It includes straight chain as well as branched chain alkyl groups.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkoxy” refers to an alkoxy group as defined above, wherein one or more hydrogen atoms are replaced by a halogen.

“Alkylthio” refers to the group “alkyl-S—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently selected from the group consisting of hydrogen, alkyl, aryl, haloalkyl, or heteroaryl; each of which may be optionally substituted.

“Amino” refers to the group —NR^{y}R^{z} wherein R^{y} and R^{z} are independently selected from the group consisting of hydrogen, alkyl, haloalkyl, aryl, or heteroaryl; each of which may be optionally substituted.

“Amidino” refers to —C(NH)(NH₂).

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Azido” refers to —N₃.

“Carbarnoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently selected from the group consisting of hydrogen, alkyl, aryl, haloalkyl, or heteroaryl; each of which may be optionally substituted.

“Carboxyl” refers to —C(O)OH.

“Carboxyl ester” refers to both —OC(O)R and —C(O)OR, wherein R is hydrogen, alkyl, cycloalkyl, heterocyclyl, aryl, heteroalkyl, or heteroaryl; each of which may be optionally substituted, as defined herein.

“Cyano” or “carbonitrile” refers to the group —CN.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Guanidino” refers to —NHC(NH)(NH₂).

“Hydrazino” refers to —NHNH₂.

“Imino” refers to a group —C(NR)R, wherein each R is alkyl, cycloalkyl, heterocyclyl, aryl, heteroalkyl, or heteroaryl; each of which may be optionally substituted, as defined herein.

“Halogen” or “halo” includes fluoro, chloro, bromo, and iodo. “Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C1-20 heteroaryl), 3 to 12 ring carbon atoms (i.e., C3-12 heteroaryl), or 3 to 8 carbon ring atoms (i.e., C3-8 heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e. the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g. 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Hydroxy” or “hydroxyl” refers to the group —OH.

“Oxo” refers to the group (═O) or (O).

“Nitro” refers to the group —NO₂.

“Sulfonyl” refers to the group —S(O)₂R, where R is alkyl, haloalkyl, heterocyclyl, cycloalkyl, heteroaryl, or aryl. Examples of sulfonyl are methylsulfonyl, ethylsulfonyl, phenylsulfonyl, and toluenesulfonyl.

“Alkylsulfonyl” refers to the group —S(O)₂R, where R is alkyl.

“Sulfonic acid” refers to the group —SO₃H.

“Alkylsulfinyl” refers to the group —S(O)R, where R is alkyl.

“Thiocyanate” refers to the group —SCN.

“Thiol” refers to the group —SH.

“Thioxo” or “thione” refer to the group (═S) or (S).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g. arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

The term “co-crystal” as used herein refers to a single-phase crystalline material of two or more different atoms, ions or molecules. Examples of co-crystals include anhydrates, hydrates, solvates, and clathrates. The components of a co-crystal typically associate via one or more non-covalent interactions such as hydrogen bonding, ionic interactions, van der Waals interactions, and pi-pi interactions. In certain embodiments, the co-crystal of a particular compound can have an improved property as compared to the free form of that compound. In various embodiments, the improved property may include increased solubility, increased dissolution, increased bioavailability, increased dose response, decreased hygroscopicity, a crystalline form of a normally amorphous compound, a crystalline form of a difficult to salt or unsaltable compound, decreased form diversity, or more desired morphology.

The term “complex” as used herein with reference to a compound described herein (e.g. Compound I as a “phosphate complex”) includes a co-crystal and a salt comprising that compound. It should be noted that the difference between a co-crystal and a salt lies merely in the transfer of a proton. The transfer of protons from one component to another in a crystal is dependent on the environment. For this reason, crystalline co-crystals and salts may be thought of as two ends of a proton-transfer spectrum, where an absence of proton transfer exists for co-crystals at one end and where proton transfer has occurred in a salt at the other end.

The term “salt” of a given compound refers to acid addition salts and base addition salts. Acid addition salts include, for example, salts with inorganic acids and salts with organic acids. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri- cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like. In one embodiment, the salt can be a phosphoric acid salt.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

In addition, abbreviations as used herein have respective meanings as follows:

| | |
|---|---|
| d | doublet |
| DMSO | dimethylsulfoxide |
| i-Pr | isopropyl |
| m | multiplet |
| MHz | megahertz |
| NMR | nuclear magnetic resonance |
| s | singlet |
| t | triplet |
| TFA | trifluoroacetic acid |
| THF | tetrahydrofuran |

### Process

As described generally above, the disclosure provides in some embodiments a process for making compounds of formula I and II. In another embodiment, the disclosure provides processes for making intermediates for the compounds of formula I and II as shown in the Scheme below.

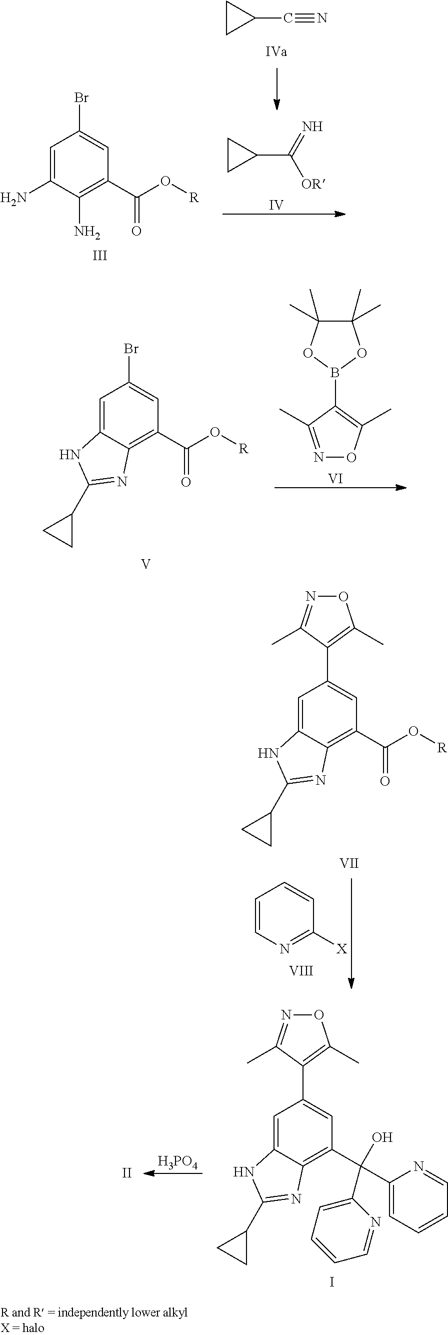

In one embodiment, this disclosure provides a process for making a compound of formula I, named (2-cyclopropyl-6-(3,5-dimethylisoxazol-4-yl)-1H-benzo[d]imidazol-4-yl)di(pyridin-2-yl)methanol:

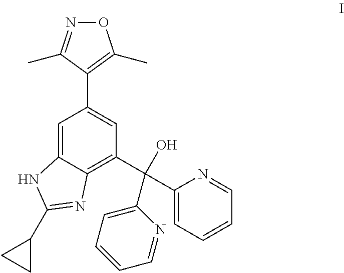 [SMILES: Cc1noc(C)c1-c1cc(C(O)(c2ccccn2)c2ccccn2)c2nc(C3CC3)[nH]c2c1]

or a salt or co-crystal thereof.

In another embodiment, this disclosure provides a process for preparation of a compound of formula I, named (2-cyclopropyl-6-(3,5-dimethylisoxazol-4-yl)-1H-benzo[d]imidazol-4-yl)di(pyridin-2-yl)methanol:

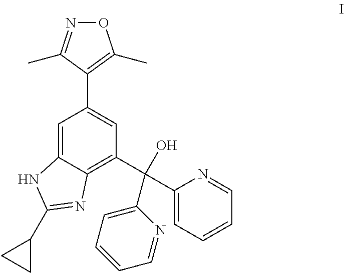 [SMILES: Cc1noc(C)c1-c1cc(C(O)(c2ccccn2)c2ccccn2)c2nc(C3CC3)[nH]c2c1]

or a salt or co-crystal thereof, comprising:
(a) contacting a compound of formula III or a salt or co-crystal thereof, with a compound of formula I or a salt or co-crystal thereof:

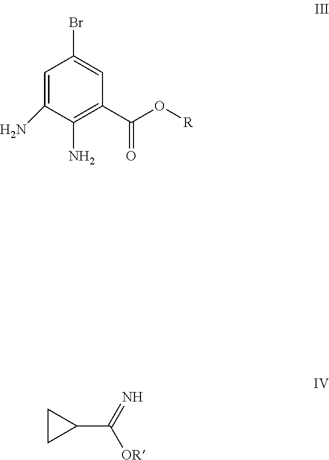

to provide a compound of formula V:

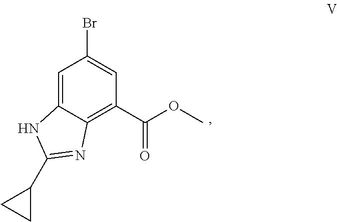 [SMILES: COC(=O)c1cc(Br)cc2[nH]c(C3CC3)nc12]

or a salt or co-crystal thereof, wherein R and R′ are indendently C₁₋₆ alkyl;
(b) contacting the compound of formula V or a salt or co-crystal thereof with a compound of formula VI:

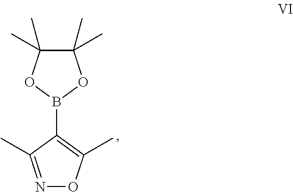

or a salt or co-crystal thereof, to provide a compound of formula VII:

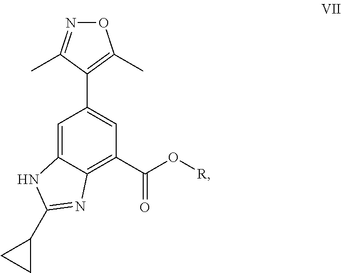

or a salt or co-crystal thereof; and
(c) contacting the compound of formula VII or a salt or co-crystal thereof with a compound of formula VIII:

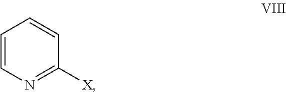

or a salt or co-crystal thereof, wherein X is halo, to provide the compound of formula I.

In another embodiment, step (a) comprises a solvent. The non-limiting examples of the solvent in step (a) include alcoholic solvents or a mixture of alcoholic solvents. In one embodiment, the solvent in step (a) is a C₁₋₆ alcohol or a mixture thereof.

In one embodiment, step (a) further comprises a temperature of about 20° C. to about 65° C. In one embodiment, step (a) further comprises a temperature from about 45° C. to about 55° C.

In one embodiment, step (b) comprises a base. The non-limiting examples of the base in step (b) include potassium carbonate, sodium carbonate, cesium carbonate, potassium bicarbonate, sodium bicarbonate, sodium acetate, potassium acetate, a dibasic phosphate or a tribasic phosphate. In one embodiment, the base in step (b) is sodium carbonate.

In one embodiment, step (b) further comprises a Pd(0) or a Pd(II) catalyst. The non-limiting examples of the Pd(0) catalyst in step (b) include tris(dibenzylideneacetone)dipalladium(0) and bis(tri-tent-butylphosphine)palladium(0). The non-limiting examples of the Pd(II) catalyst include dichloro bis(tert-butylphenylphosphoine)palladium(II), palladium acetate, [1,1′-bis(diphenylphosphino)ferrocene]dichloropalladium (II) complex with dichloromethane, and [1,3-bis(2,6-diisopropylphenyl)imidazole-2-ylidene](3-chloropyridyl) palladium(II)dichloride. In one embodiment, the Pd(II) catalyst is bis(di-tert-butyl(4-dimethylaminophenyl)phosphine)dichloropalladium(II) (PdCl₂(amphos)₂).

In one embodiment, step (b) further comprises a solvent. The non-limiting examples of the solvent in step (b) include tetrahydrofuran, 2-methyltetrahydrofuran, n-propanol, toluene, N,N-dimethylformamide, N,N-dimethylacetamide, dimethoxyethane, isopropyl acetate, n-butanol, t-amyl alcohol, methanol, ethanol, isopropanol, acetone, methyl ethyl ketone, ethyl acetate, and N-methyl-2-pyrrolidone, or a combination thereof with water. In one embodiment, the solvent in step (b) is 2-methyltetrahydrofuran and water.

In one embodiment, step (b) further comprises a temperature of from about 40° C. to about 100° C. In one embodiment, step (b) further comprises a temperature from about 55° C. to about 65° C.

In one embodiment, step (c) comprises a metallating reagent. The non-limiting examples of the metallating agent include alkyl lithium, alkyl magnesium halide, or a combination thereof with lithium halide, magnesium halide or zinc halide. In one embodiment, the alkyllithium in step (c) is n-butyl lithium or hexyllithium. In another embodiment, the alkyl magnesium halide in step (c) is isopropylmagnesium chloride. In another embodiment, the metallating agent in step (c) is isopropylmagnesium chloride/lithium chloride.

In one embodiment, step (c) further comprises a solvent. The non-limiting examples of the solvent in step (c) include tetrahydrofuran, 2-methyltetrahydrofuran, cyclopentyl methyl ether, t-butyl methyl ether, toluene, dichloromethane, or a combination thereof with tetrahydrofuran. In one embodiment, the solvent in step (c) is tetrahydrofuran.

In one embodiment, step (c) further comprises a temperature of from about −20° C. to about 65° C. In one embodiment, step (c) further comprises a temperature from about 0° C. to about 30° C.

In one embodiment, step (c) optionally comprises N,O-bis(trimethylsilyl)acetamide and trimethylchlorosilane.

In one embodiment, this disclosure provides compounds of formula III, V or VII, wherein R is methyl or ethyl. In another embodiment, this disclosure provides compounds of formula IV wherein R is methyl, ethyl or iso-propyl. In another embodiment, this disclosure provides compounds of formula VIII wherein X is bromo or iodo.

In one embodiment, this disclosure provides a process for preparation of a compound of formula IV:

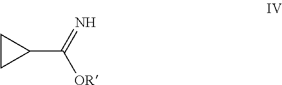

wherein R′ is C₁₋₆ alkyl, comprising treating cyanocyclopropane with acetyl chloride. The process comprises a solvent. The non-limiting examples of the solvent for the preparation of compound of formula IV include ethanol or a mixture of ethanol with other organic solvents such as methanol, isopropanol, ethyl acetate, t-butyl methyl ether, dioxane, n-propanol, and isopropanol acetate. In one embodiment, the solvent is ethanol. The process for the preparation of compound of formula IV further comprises a temperature of about 10° C. to about 70° C. In one embodiment, the temperature is from about 15° C. to about 40° C.

In one embodiment, this disclosure provides a process for preparation of a compound of formula II, named (2-cyclopropyl-6-(3,5-dimethylisoxazol-4-yl)-1H-benzo[d]imidazol-4-yl)di(pyridin-2-yl)methanol phosphate complex, comprising the process for making a compound of formula I comprising steps (a), (b) and (c) as described herein and further comprising step (d) comprising contacting the compound of formula I with phosphoric acid to provide the compound of formula II.

In one embodiment, step (a) comprises a solvent. The non-limiting examples of the solvent in step (a) include alcoholic solvents or a mixture of alcoholic solvents. In one embodiment, the solvent in step (a) is a C₁₋₆ alcohol or a mixture thereof.

In one embodiment, step (a) further comprises a temperature from about 20° C. to about 65° C. In one embodiment, step (a) comprises a temeperature from about 45° C. to about 55° C.

In one embodiment, step (b) comprises a base. The non-limiting examples of the base in step (b) include potassium carbonate, sodium carbonate, cesium carbonate, potassium bicarbonate, sodium bicarbonate, sodium acetate, potassium acetate, a dibasic phosphate or a tribasic phosphate. In one embodiment, the base in step (b) is sodium carbonate.

In one embodiment, step (b) further comprises a a Pd(0) or a Pd(II) catalyst. The non-limiting examples of the Pd(0) catalyst in step (b) include tris(dibenzylideneacetone)dipalladium(0) and bis(tri-tent-butylphosphine)palladium(0). The non-limiting examples of the Pd(II) catalyst in step (b) include dichloro bis(tert-butylphenylphosphoine)palladium(II), palladium acetate, [1,1′-bis(diphenylphosphino)ferrocene]dichloropalladium(II) complex with dichloromethane, and [1,3-bis(2,6-diisopropylphenyl)imidazole-2-ylidene](3-chloropyridyl) palladium(II)dichloride. In one embodiment, the Pd(II) catalyst in step (b) is bis(di-tert-butyl(4-dimethylaminophenyl)phosphine)dichloropalladium(II) (PdCl₂(amphos)₂).

In one embodiment, step (b) further comprises a solvent. The non-limiting examples of the solvent include tetrahydrofuran, 2-methyltetrahydrofuran, n-propanol, toluene, N,N-dimethylformamide, N,N-dimethylacetamide, dimethoxyethane, isopropyl acetate, n-butanol, t-amyl alcohol, methanol, ethanol, isopropanol, acetone, methyl ethyl ketone, ethyl acetate, and N-methyl-2-pyrrolidone or a combination thereof with water. In one embodiment, the solvent in step (b) is 2-methyltetrahydrofuran and water.

In one embodiment, step (b) further comprises a temperature from about 40° C. to about 100° C. In one embodiment, the temperature in step (b) is from about 55° C. to about 65° C.

In one embodiment, step (c) comprises a metallating reagent. The non-limiting examples of the metallating agent include alkyl lithium, alkyl magnesium halide, or a combination thereof with lithium halide, magnesium halide or zinc halide. In one embodiment, the alkyllithium in step (c) is n-butyl lithium or hexyllithium. In another embodiment, the alkyl magnesium halide in step (c) is isopropylmagnesium chloride. In another embodiment, the metallating agent in step (c) is isopropylmagnesium chloride/lithium chloride.

In one embodiment, step (c) further comprises a solvent. The non-limiting examples of the solvent in step (c) include tetrahydrofuran, 2-methyltetrahydrofuran, cyclopentyl methyl ether, t-butyl methyl ether, toluene, dichloromethane, or a combination thereof with tetrahydrofuran. In one embodiment, the solvent in step (c) is tetrahydrofuran.

In one embodiment, step (c) further comprises a temperature from about −20° C. to about 65° C. In one embodiment, the temperature in step (c) is from about 0° C. to about 30° C.

In one embodiment, step (c) optionally comprises N,O-bis(trimethylsilyl)acetamide and trimethylchlorosilane.

In one embodiment, step (d) comprises a solvent. The non-limiting examples of the solvent in step (d) include methanol, ethanol, isopropanol, acetone, toluene, tetrahydrofuran, 2-methyltetrahydrofuran, acetonitrile, isopropanol acetate, dichloromethane, dimethyl sulfoxide, N,N-dimethylformamide, N,N-dimethylacetamide, ethyl acetate, N-methyl-2-pyrrolidone, n-propanol, n-butanol, methyl ethyl ketone, and water or a mixture thereof.

In one embodiment, step (d) further comprises a temperature from about −20° C. to about 70° C. In one embodiment, step (d) comprises a temperature from about 50° C. to about 65° C.

Similarly, phosphate salts or co-crystals of Compound I are prepared using more than 1 equivalent of phosphoric acid. For example, Compound I.2H₃PO₄ or Compound I.3H₃PO₄ salts or co-crystals are prepared by using 2 or 3 equivalents of phosphoric acid respectively.

In one embodiment, this disclosure provides a process for preparation of a compound of formula VII:

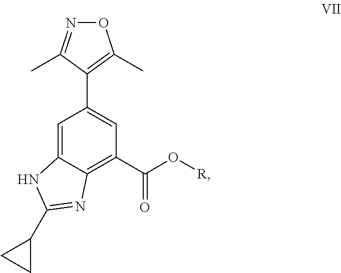

or a salt or co-crystal thereof, comprising contacting a compound of formula V or a salt or co-crystal thereof with a compound of formula VI or a salt or co-crystal thereof:

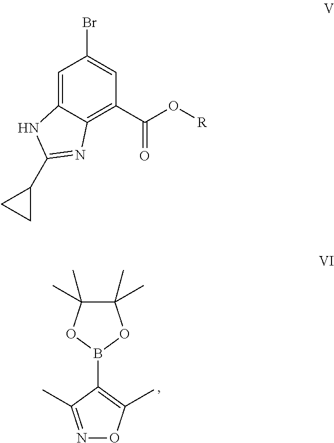

to provide the compound of formula VII or a salt or co-crystal thereof, wherein R is C₁₋₆ alkyl. Other boronate esters substantially similar to compound VI may also be used in place of compound VI in the process described above, for example 4-(1,3,2-dioxaborolan-2-yl)-3,5-dimethyl-isoxazole.

In one embodiment, the process for preparation of a compound of formula VII comprises a base. The non-limiting examples of the base include potassium carbonate, sodium carbonate, cesium carbonate, potassium bicarbonate, sodium bicarbonate, sodium acetate, potassium acetate, a dibasic phosphate or a tribasic phosphate. In one embodiment, the base is sodium carbonate.

In one embodiment, the process for preparation of a compound of formula VII further comprises a a Pd(0) or a Pd(II) catalyst. The non-limiting examples of the Pd(0) catalyst include tris(dibenzylideneacetone)dipalladium(0) and bis(tri-tert-butylphosphine)palladium(0). The non-limiting examples of the Pd(II) catalyst include dichloro bis(tert-butylphenylphosphoine)palladium(II), palladium acetate, [1,1′-bis(diphenylphosphino)ferrocene]dichloropalladium(II) complex with dichloromethane, and [1,3-bis(2,6-diisopropylphenyl)imidazole-2-ylidene](3-chloropyridyl) palladium(II)dichloride. In one embodiment, the Pd(II) catalyst is bis(di-tert-butyl(4-dimethylaminophenyl)phosphine)dichloropalladium(II) (PdCl₂(amphos)₂).

In one embodiment, the process for preparation of a compound of formula VII further comprises a solvent. The non-limiting examples of the solvent include tetrahydrofuran, 2-methyltetrahydrofuran, n-propanol, toluene, N,N-dimethylformamide, N,N-dimethylacetamide, dimethoxyethane, isopropyl acetate, n-butanol, t-amyl alcohol, methanol, ethanol, isopropanol, acetone, methyl ethyl ketone, ethyl acetate, and N-methyl-2-pyrrolidone, or a combination thereof with water. In one embodiment, the solvent for preparation of a compound of formula VII is 2-methyltetrahydrofuran and water.

In one embodiment, the process for preparation of a compound of formula VII further comprises a temperature from about 40° C. to about 100° C. In one embodiment, the temperature is from about 55° C. to about 65° C.

In one embodiment, this disclosure provides a process for preparation of a compound of formula V:

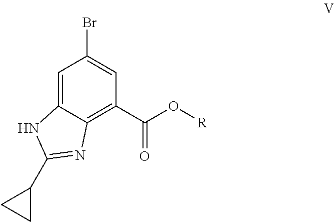

or a salt or co-crystal thereof, comprising contacting a compound of formula IX or a salt or co-crystal thereof with cyclopropylcarboxaldehyde:

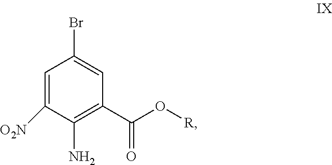

to provide the compound of formula V or a salt or co-crystal thereof, wherein R is C₁₋₆ alkyl.

In one embodiment, the process for preparation of a compound of formula V further comprises a solvent. Non-limiting examples of the solvent include N,N-dimethylformamide, N,N-dimethylacetamide, dimethylsulfoxide, N-methyl-2-pyrrolidone, or acetonitrile, in combination with water, an alcoholic solvent (e.g., methanol), or a combination thereof. In one embodiment, the solvent for preparation of a compound of formula V is N,N-dimethylacetamide in combination with water and methanol.

In one embodiment, the process for preparation of a compound of formula V further comprises a temperature from about 20° C. to about 100° C. In one embodiment, the temperature is from about 70° C. to about 90° C.

In one embodiment, the process for preparation of a compound of formula V further comprises a reducing agent. In one embodiment, the reducing agent is sodium dithionite.

### EXAMPLES

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of compounds described herein, may be accomplished as described in the following examples. If available, reagents may be purchased commercially, e.g. from Sigma Aldrich or other chemical suppliers. Unless otherwise noted, the starting materials for the following reactions may be obtained from commercial sources.

Examples related to the present invention are described below. In most cases, alternative techniques can be used. The examples are intended to be illustrative and are not limiting or restrictive to the scope of the invention. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

¹H Nuclear magnetic resonance (NMR) spectroscopy was carried out using a Bruker 300 MHz probe. The spectra were acquired in the stated solvent at around room temperature unless otherwise stated. In all cases, NMR data were consistent with the proposed structures. Flash chromatography was performed using 40-63 μm (230-400 mesh) silica gel from Silicycle following analogous techniques to those disclosed in Still, W. C.; Kahn, M.; and Mitra, M. Journal of Organic Chemistry, 1978, 43, 2923.

### Compound Preparation

Where the preparation of starting materials is not described, these are commercially available, known in the literature, or readily obtainable by those skilled in the art using standard procedures. Where it is stated that compounds were prepared analogously to earlier examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is a Biotage Initiator. The actual power supplied varies during the course of the reaction in order to maintain a constant temperature.

### Example 1

### Synthesis of [(2-cyclopropyl-6-(3,5-dimethylisoxazol-4-yl)-1H-benzo[d]imidazol-4-yl)di(pyridin-2-yl)methanol phosphate salt or co-crystal

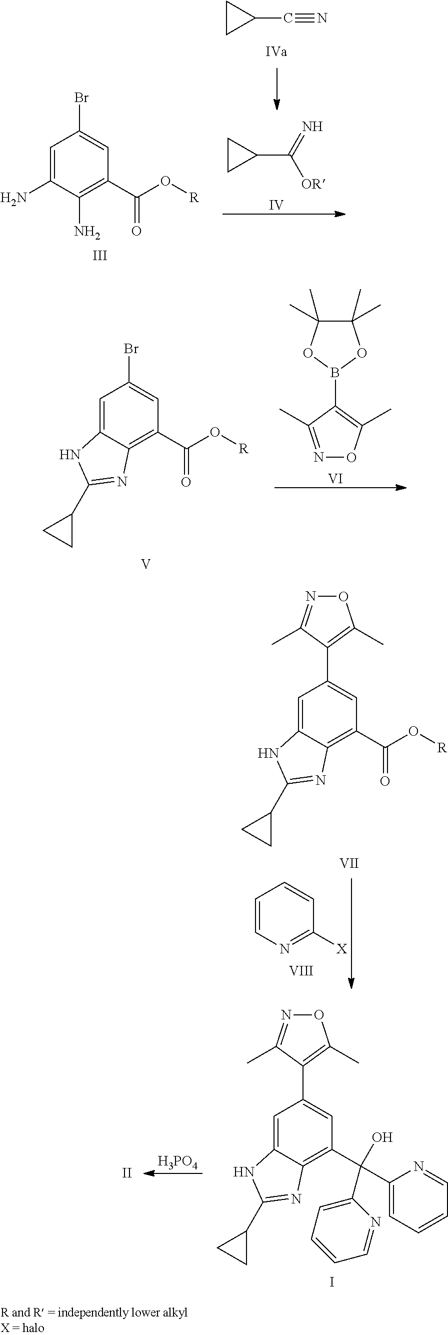

### Step 1: Conversion of III to V

a. Conversion of IVa to HCl Salt of IV

Acetyl acid chloride (5.8 equivalents) was charged slowly to a reactor containing ethanol (1.1 volumes). The mixture was adjusted to about 3° C. and agitated for about 30 minutes, followed by addition of compound IVa (1.0 equivalent). The mixture was agitated at about 20° C. until the reaction was complete, and concentrated under reduced pressure; the concentrate was further co-evaporated with heptane (3×1.3 volumes). The resulting slurry was filtered and the filter cake rinsed with heptane (0.6 volumes) and dried to afford HCl salt of compound IV (R′=ethyl).

Alternatively, a mixture of ethanol with other organic solvents such as methanol, isopropanol, ethyl acetate, t-butyl methyl ether, dioxane, n-propanol, and isopropanol acetate can also be used in this reaction. Also, the reaction temperature can range from about 10° C. to about 70° C.

b. Conversion of IV to V

The HCl salt of compound IV (R′=ethyl, 1.5 equivalents), compound III (R=methyl, 1.0 equivalent) and methanol (5.1 volumes) were charged to a reactor. The mixture was agitated at about 50° C. until the reaction was complete. t-Butyl methyl ether (10.8 volumes) and 5% sodium bicarbonate solution (7.4 volumes) were charged to the reaction mixture. The organic layer was separated and combined with t-butyl methyl ether (6.8 volumes), and washed with 9% sodium chloride solution (4.8 volumes) and water (5.0 volumes), respectively. The organic layer was concentrated under reduced pressure and co-evaporated with t-butyl methyl ether (5.4 volumes). t-Butyl methyl ether (1.4 volumes) was added to the concentrate and agitated at about 50° C. for about 30 minutes, followed by addition of heptanes (10.2 volumes). The mixture was cooled to about 20° C. over a minimum of about 2 hours and agitated for about 1 hour. The slurry was filtered and the filter cake rinsed with a mixture of heptanes (2.9 volumes) and t-butyl methyl ether (0.7 volume) and dried to afford compound V (R=methyl, 110 g). ¹H NMR (400 MHz, CDCl₃): δ 10.20 (s 1H), 7.99 (m, 2H), 4.00 (S, 3H), 2.10 (m, 1H), 1.12(m, 4H).

Alternatively, other alcoholic solvents such as ethanol or a mixture of alcoholic solvents can also be used in this reaction. Also, the reaction temperature can range from about 20° C. to about 65° C.

Alternatively, compound V (R=methyl) was isolated directly from the reaction mixture by adding acetonitrile (5.1 volumes) and water (7.0 volumes) at about 20° C., followed by seeding with compound V and addition of water (3.0 volumes) and 0.4% sodium bicarbonate solution (6.0 volumes). The resulting slurry was filtered; the filter cake was washed with water (6.0 volumes) and heptanes (4.4 volumes), and dried to afford compound V (107 g).

Step 2: Conversion of V to VII

Compound V (R=methyl, 1.0 equivalent), compound VI (1.1 equivalents) and 2-methyltetrahydrofuran (11.6 volumes) were charged to a reactor and the reactor is inerted. Degassed sodium carbonate solution (sodium carbonate 1.7 equivalents, water 6.0 volumes) and bis(di-tert-butyl(4-dimethylaminophenyl)phosphine)dichloropalladium(II) (0.02 equivalent) were charged to the reactor and the mixture was degassed. The reaction mixture was agitated at about 60° C. until the reaction was complete. The reaction mixture was cooled to about 20° C. and 2-methyltetrahydrofuran (11.6 volumes) and 20% potassium phosphate tribasic solution (5.0 volumes) were charged. After layer separation, N-acetyl-L-cysteine (0.45 equivalent) was charged to the organic layer and the mixture was agitated at about 45° C. for about 15 hours. Upon cooling to about 20° C., the mixture was washed with 20% potassium phosphate tribasic solution (5.0 volumes); the separated organic layer was further washed with water (3×5.0 volumes) at about 40° C. The organic layer was concentrated under reduced pressure and further co-evaporated with 2-methyltetrahydrofuran (2×7.0 volumes) and with t-butyl methyl ether (4.1 volumes). t-Butyl methyl ether (4.1 volumes) was charged to the concentrate and the mixture was agitated at about 55° C. for approximately 1 hour. Heptanes (8.8 volumes) were charged to the mixture while maintaining the temperature at about 55° C. The resulting slurry was agitated for about 30 minutes and cooled to about 20° C. and agitated for about 1 hour. The slurry was filtered and the filter cake rinsed with heptanes (2.9 volumes) and dried under reduced pressure to afford compound VII (R=methyl, 19 g). ¹H NMR (400 MHz, DMSO-d6): δ 12.60 (s, 1H), 7.80 (s, 1H), 7.65 (s, 1H), 4.00 (s, 3H), 2.40 (m, 4H), 2.20 (s, 3H), 1.10 (m, 4H).

Alternatively, other bases such as potassium carbonate, cesium carbonate, potassium bicarbonate, sodium bicarbonate, sodium acetate, potassium acetate, a dibasic phosphate or a tribasic phosphate can be used. Also, other Pd(II) catalysts such as dichloro bis(tert-butylphenylphosphoine)palladium(II), palladium acetate, [1,1′-bis(diphenylphosphino)ferrocene]dichloropalladium(II) complex with dichloromethane, and [1,3 -bis(2,6-diisopropylphenyl)imidazole-2-ylidene](3-chloropyridyl) palladium(II)dichloride can be used. Also, other solvents such as tetrahydrofuran, 2-methyltetrahydrofuran, n-propanol, toluene, N,N-dimethylformamide, N,N-dimethylacetamide, dimethoxyethane, isopropyl acetate, n-butanol, t-amyl alcohol, methanol, ethanol, isopropanol, acetone, methyl ethyl ketone, ethyl acetate, and N-methyl-2-pyrrolidone, or a combination thereof with water can be used. Also, the reaction temperature can range from about 40° C. to about 100° C.

Step 3: Conversion of VII to I

Compound VIII (X=Br, 4.5 equivalents) and tetrahydrofuran (2.3 volumes) were charged to a reactor; the reaction mixture was degassed and adjusted to about 20° C. 14% i-PrMgCl.LiCl solution in THF (4.5 equivalents) was charged to the reactor and the mixture was agitated at about 20° C. until the reaction was complete. The reaction mixture is cooled to around −20° C., and a mixture of degassed solution of compound VII (1.0 equivalent) in tetrahydrofuran (5.6 volumes) with N,O-bis(trimethylsilyl)acetamide (1.0 equivalent) and trimethylchlorosilane (0.03 equivalent) was charged to the reactor. The reaction mixture was agitated at about 10° C. until the reaction was complete. The reaction mixture was transferred to a mixture of water (35 volumes), ammonium chloride (35 equivalents), aqueous HCl (37%, 0.6 volume) and 2-methyltetrahydrofuran (17.4 volumes), while maintaining the temperature at not more than 35° C. The organic layer was separated and washed with water (2×10 volumes). The organic layer was concentrated under reduced pressure, and co-evaporated with ethyl acetate three times (5.5 volumes, 5.5 volumes, and 11.1 volumes, respectively). To the resulting slurry, pyridine (0.1 volume), tetrahydrofuran (0.1 volume) and water (0.05 volume) were added. The slurry was agitated at about 50° C. for approximately 30 minutes, and then cooled to about 20° C. and agitated for about 1 hour. The slurry was filtered and the filter cake rinsed with ethyl acetate (2.2 volumes). The filter cake and methanol (6.3 volumes) were charged to the reactor and agitated at about 60° C. for approximately 3 hours. Upon cooling to about 20° C. over a minimum of 1 hour, followed by additional a minimum of 1 hour agitation at about 20° C., the slurry was filtered and the filter cake rinsed with methanol (1.3 volumes) and dried under reduced pressure to afford compound I (30 g). ¹H NMR (400 MH, DMSO-d₆/TFA-dⱼ): δ 8.54 (m, 2H), 7.89 (m, 2H), 7.66 (m, 2H), 7.58 (d, 1H), 7.37 (m, 2H), 7.04 (d, 1H), 2.61 (m, 1H), 2.30 (s, 3H), 2.10 (s, 3H), 1.28-1.39 (m, 4H).

Alternatively, the reaction can also be conducted in the absence of N,O-bis(trimethylsilyl)acetamide and trimethylchlorosilane.

Alternatively, other metallating agents such as alkyl lithium, alkyl magnesium halide, or a combination thereof with lithium halide, magnesium halide or zinc halide can be used. Also, other solvents such as tetrahydrofuran, 2-methyltetrahydrofuran, cyclopentyl methyl ether, t-butyl methyl ether, toluene, dichloromethane, or a combination thereof with tetrahydrofuran can be used. Also, the reaction temperature can range from about −20° C. to about 65° C.

Step 4: Conversion of I to II

Compound I (1.0 equivalent) and methanol (12.6 volumes) were charged to a reactor; the mixture was agitated at about 60° C. until a solution is achieved. A solution of phosphoric acid (85% wt) (1.06 equivalents) in methanol (0.6 volumes) was charged followed by a methanol rinse (2.5 volumes). The reaction contents were seeded with compound II and agitated at about 55° C. for approximately 6 hours. Ethyl acetate (27.2 volumes) was charged at about 55° C. over approximately 4 hours. The reaction mixture was agitated at about 55° C. for a minimum of 12 hours, and then cooled to about 20° C. over about 2 hours and agitated for about 1 hour. The slurry was filtered and the filter cake rinsed with ethyl acetate (2.2 volumes) and n-heptane (2.9 volumes), respectively, and dried to afford compound II (10 g). ¹H NMR (400 MHz, DMSO-d₆): δ 8.50 (d, 2H), 7.80 (t, 2H), 7.62 (d, 2H), 7.38 (s, 1H), 7.30 (t, 2H), 6.70 (m, 2H), 2.27 (s, 4H), 2.08 (s, 3H), 1.00 (m, 4H).

Alternatively, phosphoric acid with various concentrations can be used in this reaction. For example, Compound I.2H₃PO₄ or Compound I.3H₃PO₄ salts are prepared by using 2 or 3 equivalents of phosphoric acid respectively. Also, other solvents such as methanol, ethanol, isopropanol, acetone, toluene, tetrahydrofuran, 2-methyltetrahydrofuran, acetonitrile, isopropanol acetate, dichloromethane, dimethyl sulfoxide, N,N-dimethylformamide, N,N-dimethylacetamide, N-methyl-2-pyrrolidone, n-propanol, n-butanol, and water or a mixture thereof can be used. Also, the reaction temperature can range from about −20° C. to about 70° C.

Also, other salts can be prepared using acids such as hydrochloric acid, sulfuric acid, p-toluenesulfuric acid, benzenesulfonic, methanesulfuric acid, tatric acid, and oxalic acid.

The methods and variances described herein as representative of preferred embodiments are exemplary and are not intended as limitations on the scope of the disclosure. Thus, additional embodiments are within the scope of the disclosure and within the following claims.

### Example 2

### Synthesis of Compound V

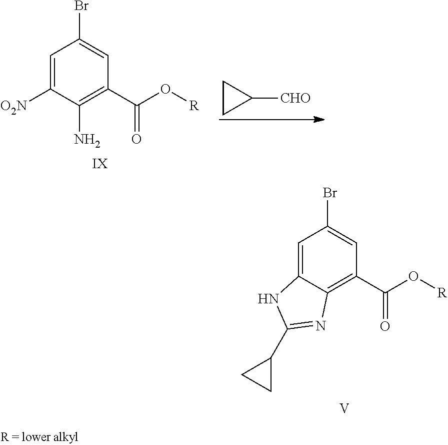

To an agitated solution of compound IXa (R=methyl) (1.0 equivalent) in dimethylacetamide (10.7 volumes) at about 20° C., sodium dithionite (3.0 equivalents) and water (0.5 volumes) were added. The resulting slurry was agitated at about 80° C. for approximately 2 hours prior to the addition of methanol (6.3 volumes). A solution of cyclopropanecarbaldehyde (1.2 equivalents) in methanol (6.3 volumes) was added to the reaction mixture at about 80° C. over approximately 2 hours. After the addition was complete, the reaction mixture was agitated at about 75-80° C. for approximately 4 hours, and then adjusted to about 20° C. and concentrated under vacuum to remove methanol. Water (10.0 volumes) was slowly added to the concentrate. The pH of the resulting mixture was adjusted to approximately 7 with a solution of sodium bicarbonate in water (7.5 volumes), followed by addition of water (10.0 volumes). The resulting slurry was agitated at about 20° C. for approximately 24 hours, and filtered. The wet filter cake was rinsed with water twice (10.0 volumes each), and dried to afford Va (R=methyl) (56 g).

What is claimed is:

1. A process for preparation of a compound of formula VII: 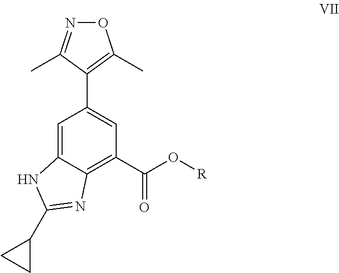
or a salt or co-crystal thereof, comprising contacting a compound of formula V or a salt or co-crystal thereof, wherein R is C₁₋₆ alkyl, with a compound of formula VI or a salt or co-crystal thereof: 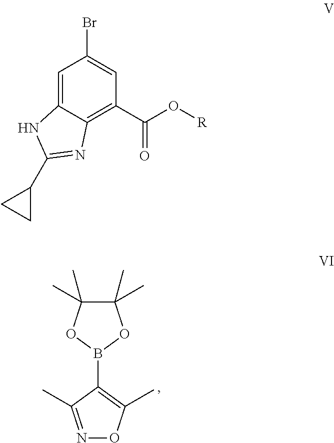
to provide the compound of formula VII or a salt or co-crystal thereof.

2. The process of claim 1, wherein the process comprises a base.

3. The process of claim 2, wherein the base is a carbonate, bicarbonate, acetate or phosphate.

4. The process of claim 3, wherein the base is potassium carbonate, sodium carbonate, cesium carbonate, potassium bicarbonate, sodium bicarbonate, sodium acetate, potassium acetate, a dibasic phosphate or a tribasic phosphate.

5. The process of claim 4, wherein the base is sodium carbonate.

6. The process of claim 1, wherein the process comprises a Pd(0) or a Pd(II) catalyst.

7. The process of claim 1, wherein the process comprises a solvent selected from tetrahydrofuran, 2-methyltetrahydrofuran, n-propanol, toluene, N,N-dimethylformamide, N,N-dimethylacetamide, dimethoxyethane, isopropyl acetate, n-butanol, t-amyl alcohol, methanol, ethanol, isopropanol, acetone, methyl ethyl ketone, ethyl acetate, and N-methyl-2-pyrrolidone, or a combination thereof with water.

8. The process of claim 7, wherein the solvent is 2-methyltetrahydrofuran and water.

9. The process of claim 1, wherein the process comprises a temperature from about 40° C. to about 100° C.

10. The process of claim 9, wherein the temperature is from about 55° C. to about 65° C.

11. A process for preparation of a compound of formula I: 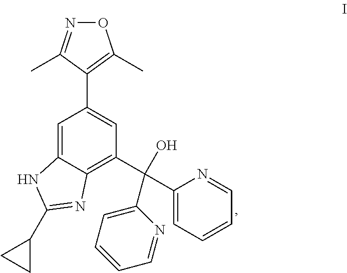 [SMILES: Cc1noc(C)c1-c1cc(C(O)(c2ccccn2)c2ccccn2)c2nc(C3CC3)[nH]c2c1]
comprising:
(a) contacting a compound of formula III or a salt or co-crystal thereof, with a compound of formula IV or a salt or co-crystal thereof: 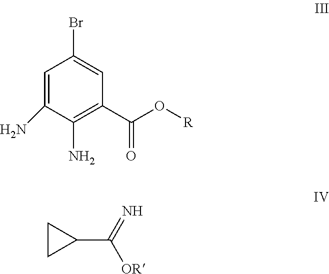
to provide a compound of formula V: 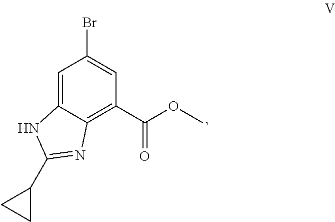 [SMILES: COC(=O)c1cc(Br)cc2[nH]c(C3CC3)nc12]
or a salt or co-crystal thereof, wherein R and R′ are indendently C₁₋₆ alkyl;
(b) contacting the compound of formula V or a salt or co-crystal thereof with a compound of formula VI: 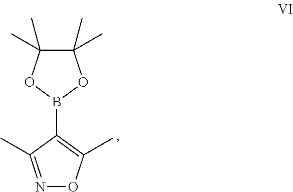
or a salt or co-crystal thereof, to provide a compound of formula VII: 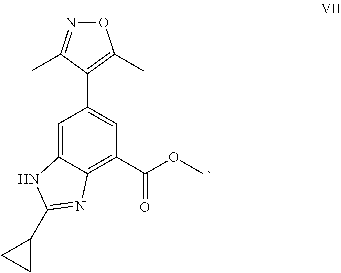 [SMILES: COC(=O)c1cc(-c2c(C)noc2C)cc2[nH]c(C3CC3)nc12]
or a salt or co-crystal thereof; and
(c) contacting the compound of formula VII or a salt or co-crystal thereof with a compound of formula VIII: 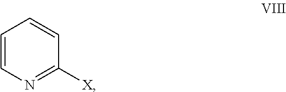
or a salt or co-crystal thereof, wherein X is halo, to provide the compound of formula I.

12. The process of claim 11, wherein step (a) comprises a solvent selected from a C₁₋₆ alcohol or a mixture thereof

13. The process of claim 11, wherein step (a) comprises a temperature from about 20° C. to about 65° C.

14. The process of claim 11, wherein step (a) comprises methanol as a solvent and a temperature from about 45° C. to about 55° C.

15. The process of claim 11, wherein step (b) comprises a base.

16. The process of claim 15, wherein the base is potassium carbonate, sodium carbonate, cesium carbonate, potassium bicarbonate, sodium bicarbonate, sodium acetate, potassium acetate, a dibasic phosphate or a tribasic phosphate.

17. The process of claim 16, wherein the base is sodium carbonate.

18. The process of claim 11, wherein step (b) comprises a Pd(0) or a Pd(II) catalyst.

19. The process of claim 11, wherein step (b) comprises a solvent selected from tetrahydrofuran, 2-methyltetrahydrofuran, n-propanol, toluene, N,N-dimethylformamide, N,N-dimethylacetamide, dimethoxyethane, isopropyl acetate, n-butanol, t-amyl alcohol, methanol, ethanol, isopropanol, acetone, methyl ethyl ketone, ethyl acetate, and N-methyl-2-pyrrolidone, or a combination thereof with water.

20. The process of claim 11, wherein step (b) comprises a temperature from about 40° C. to about 100° C.

21. The process of claim 11, wherein step b) comprises 2-methyltetrahydrofuran and water as a solvent and a temperature from about 55° C. to about 65° C.

22. The process of claim 11, wherein step (c) comprises a metallating reagent selected from alkyl lithium, alkyl magnesium halide, or a combination thereof with lithium halide, magnesium halide or zinc halide.

23. The process of claim 11, wherein step (c) comprises a solvent selected from tetrahydrofuran, 2-methyltetrahydrofuran, cyclopentyl methyl ether, t-butyl methyl ether, toluene, and dichloromethane, or a combination thereof with tetrahydrofuran.

24. The process of claim 11, wherein step (c) comprises a temperature from about −20° C. to about 65° C.

25. The process of claim 11, wherein step (c) comprises isopropylmagnesium chloride-lithium chloride as the metallating reagent, tetrahydrofuran as a solvent and a temperature from about 0° C. to about 30° C.

26. The process of claim 11, wherein step (c) optionally comprises N,O-bis(trimethylsilyl)acetamide and trimethylchlorosilane.

27. A process for preparation of (2-cyclopropyl-6-(3,5-dimethylisoxazol-4-yl)-1H-benzo[d]imidazol-4-yl)di(pyridin-2-yl)methanol phosphate complex (Formula II), comprising
(a) contacting a compound of formula III or a salt or co-crystal thereof, with a compound of formula IV or a salt or a co-crystal thereof: 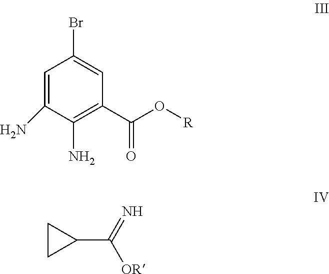
to provide a compound of formula V: 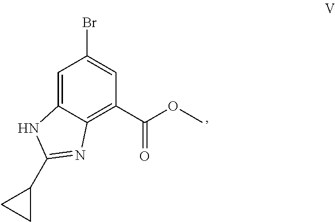 [SMILES: COC(=O)c1cc(Br)cc2[nH]c(C3CC3)nc12]
or a salt or co-crystal thereof, wherein R and R′ are indendently C₁₋₆ alkyl;
(b) contacting the compound of formula V or a salt or co-crystal thereof with a compound of formula VI: 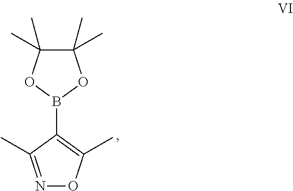
or a salt or co-crystal thereof, to provide a compound of formula VII: 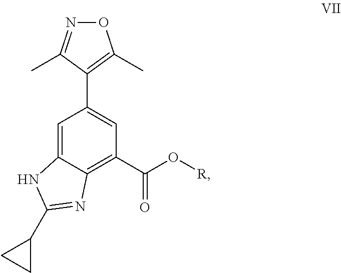
or a salt or co-crystal thereof;
(c) contacting the compound of formula VII or a salt or co-crystal thereof with a compound of formula VIII: 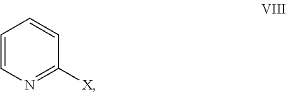
or a salt or co-crystal thereof, to provide the compound of formula I: 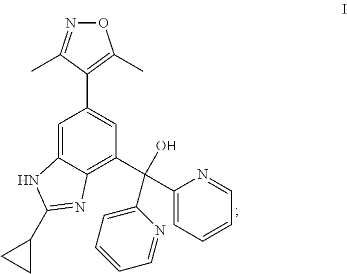 [SMILES: Cc1noc(C)c1-c1cc(C(O)(c2ccccn2)c2ccccn2)c2nc(C3CC3)[nH]c2c1]
and
(d) contacting the compound of formula I with phosphoric acid to provide the compound of formula II.

28. The process of claim 27, wherein step (a) comprises a solvent selected from a C₁₋₆ alcohol or a mixture thereof.

29. The process of claim 27, wherein step (a) comprises a temperature of about 20° C. to about 65° C.

30. The process of claim 27, wherein step (a) comprises methanol as a solvent and a temperature of about 45° C. to about 55° C.

31. The process of claim 27, wherein step (b) comprises a base.

32. The process of claim 31, wherein the base is potassium carbonate, sodium carbonate, cesium carbonate, potassium bicarbonate, sodium bicarbonate, sodium acetate, potassium acetate, a dibasic phosphate or a tribasic phosphate.

33. The process of claim 31, wherein the base is sodium carbonate.

34. The process of claim 27, wherein step (b) comprises a Pd(0) or a Pd(II) catalyst.

35. The process of claim 27, wherein step (b) comprises a solvent selected from tetrahydrofuran, 2-methyltetrahydrofuran, n-propanol, toluene, N,N-dimethylformamide, N,N-dimethylacetamide, dimethoxyethane, isopropyl acetate, n-butanol, t-amyl alcohol, methanol, ethanol, isopropanol, acetone, methyl ethyl ketone, ethyl acetate, and N-methyl-2-pyrrolidone, or a combination thereof with water.

36. The process of claim 27, wherein step (b) comprises a temperature of about 40° C. to about 100° C.

37. The process of claim 27, wherein step (b) comprises 2-methyltetrahydrofuran and water as a solvent and a temperature of about 55° C. to about 65° C.

38. The process of claim 27, wherein step (c) comprises a metallating reagent selected from alkyl lithium, alkyl magnesium halide, or a combination thereof with lithium halide, magnesium halide or zinc halide.

39. The process of claim 27, wherein step (c) comprises a solvent selected from 2-methyltetrahydrofuran, cyclopentyl methyl ether, t-butyl methyl ether, toluene, and dichloromethane, or a combination thereof with tetrahydrofuran.

40. The process of claim 27, wherein step (c) comprises a temperature of about -20° C. to about 65° C.

41. The process of claim 27, wherein step (c) comprises isopropylmagnesium chloride-lithium chloride as the metallating reagent, tetrahydrofuran as a solvent and a temperature of about 0° C. to about 30° C.

42. The process of claim 27, wherein step (c) optionally comprises N,O-bis(trimethylsilyl)acetamide and trimethylchlorosilane.

43. The process of claim 27, wherein step (d) comprises a solvent selected from methanol, ethanol, isopropanol, acetone, toluene, tetrahydrofuran, 2-methyltetrahydrofuran, acetonitrile, isopropanol acetate, dichloromethane, dimethyl sulfoxide, N,N-dimethylformamide, N,N-dimethylacetamide, ethyl acetate, N-methyl-2-pyrrolidone, n-propanol, n-butanol, methyl ethyl ketone, and water or a mixture thereof.

44. The process of claim 27, wherein step (d) comprises a temperature of about -20° C. to about 70° C.

45. The process of claim 27, wherein step (d) comprises methanol and ethyl acetate as a solvent and a temperature of about 50° C. to about 65° C.

46. A process for preparation of (2-cyclopropyl-6-(3,5-dimethylisoxazol-4-yl)-1H-benzo[d]imidazol-4-yl)di(pyridin-2-yl)methanol phosphate complex (Formula II), comprising
(a) contacting a compound of formula IX or a salt or co-crystal thereof, with cyclopropylcarbaldehyde: 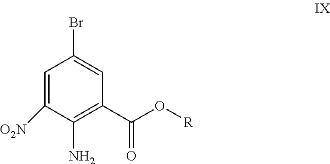
to provide a compound of formula V: 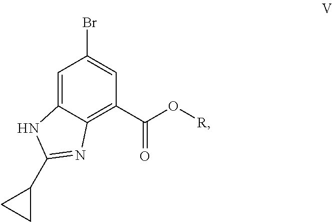
or a salt or co-crystal thereof, wherein R is C₁₋₆ alkyl;
(b) contacting the compound of formula V or a salt or co-crystal thereof with a compound of formula VI: 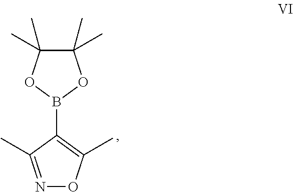
or a salt or co-crystal thereof, to provide a compound of formula VII: 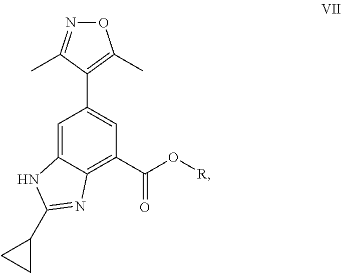
or a salt or co-crystal thereof;
(c) contacting the compound of formula VII or a salt or co-crystal thereof with a compound of formula VIII: 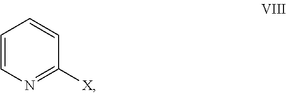
or a salt or co-crystal thereof, to provide the compound of formula I: 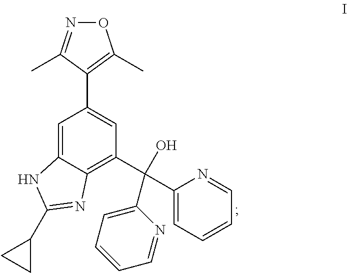 [SMILES: Cc1noc(C)c1-c1cc(C(O)(c2ccccn2)c2ccccn2)c2nc(C3CC3)[nH]c2c1]
and
(d) contacting the compound of formula I with phosphoric acid to provide the compound of formula II.

47. The process of claim 46, wherein step (a) comprises a solvent selected from N,N-dimethylformamide, N,N-dimethylacetamide, dimethylsulfoxide, N-methyl-2-pyrrolidone, and acetonitrile, in combination with water, an alcoholic solvent, or a combination thereof.

48. The process of claim 46, wherein step (a) comprises a temperature of about 20° C. to about 100° C.

49. The process of claim 46, wherein step (a) comprises N,N-dimethylacetamide in combination with water and methanol as a solvent, and a temperature of about 70° C. to about 90° C.
